(19) United States
(12) Patent Application Publication
(10) Pub. No.: US 2009/0099363 A1
(43) Pub. Date: Apr. 16, 2009

(54) PROCESS FOR THE PREPARATION OF POLYMORPHIC FORMS OF CLOPIDOGREL HYDROGEN SULFATE

(76) Inventors: Saxena Rahul, Chandigarh (IN); Verma Naresh Kumar, Panchkula (IN); Srinivasan Chidambaram Venkateswaran, Panchkula (IN); Wadhwa Lalit, Panchkula (IN)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/298,409

(22) PCT Filed: Apr. 25, 2007
(86) PCT No.: PCT/IN2007/000163

§ 371 Date: Oct. 24, 2008

(30) Foreign Application Priority Data
Apr. 27, 2006 (IN) 1075/DEL/2006
Aug. 8, 2006 (IN) 1794/DEL/2006

(51) Int. Cl. C07D 495/04

(52) U.S. Cl. 546/114

(57) ABSTRACT

The present invention relates to a novel process for the preparation of polymorphic forms of clopidogrel hydrogen sulfate, namely methyl(+)-(S)-α-(o-chlorophenyl)-6,7-dihydrothieno[3,2-c]pyridine-5(4H)-acetate hydrogen sulfate of formula (I). Particularly the present invention relates to the process for the preparation of form (I) and amorphous clopidogrel hydrogen sulfate.

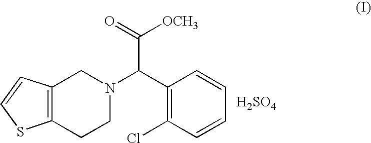

### FIELD OF THE INVENTION

The present invention relates to a novel process for the preparation of polymorphic forms of clopidogrel hydrogen sulfate. More particularly, the present invention relates to polymorphic Form I and amorphous form of methyl(+)-(S)-α-(o-chlorophenyl)-6,7-dihydrothieno[3,2-c]pyridine-5(4H)-acetate hydrogen sulfate of the Formula I

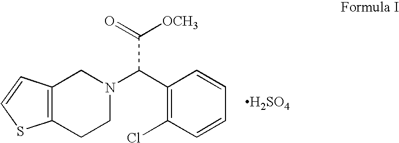

### BACKGROUND OF THE INVENTION

Clopidogrel chemically known as methyl(+)-(S)-α-(o-chlorophenyl)-6,7-dihydrothieno[3,2-c]pyridine-5(4H)-acetate, has the following structural formula:

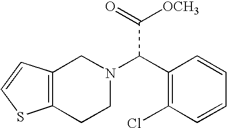

Clopidogrel is a known blood platelet aggregation inhibitory and antithrombotic pharmaceutical active ingredient. Platelet inhibiting activity of Clopidogrel makes it an effective drug for reducing the incidence of ischemic strokes, heart attack due to vascular diseases such as atherosclerosis. By inhibiting platelet aggregation, Clopidogrel reduces the chance of arterial blockage, thus preventing strokes and heart attack.

(±) Clopidogrel was first disclosed in U.S. Pat. No. 4,529,596 which discloses a racemic mixture of clopidogrel and process for preparing such a mixture by the reaction of thienopyridine derivative with a chloro compound in solvents like dimethyl formamide, alcohols and ethyl acetate in the presence of metal carbonates.

U.S. Pat. No. 4,847,265 reports the formation of (S)-(+)-Clopidogrel hydrogen sulfate for the first time, with melting point of 184° C., but was however, completely silent in disclosing the crystal form of the product.

U.S. Pat. Nos. 5,132,435; 6,258,961; 6,215,005; and 6,180,793, which are hereby incorporated by reference in their entirety, describe the methods that can be used to prepare clopidogrel hydrogen sulfate.

Active pharmaceutical ingredients and their salts are purified and isolated by crystallization from an appropriate solvent during the final step in the synthetic process. A large number of factors can influence crystal nucleation and growth during this process, including the composition, the crystallization medium and the processes used to generate super saturation and promote crystallization. The most notable variables of composition and processing are solvent/solvent combinations, degree of super saturation, pH value, heating rate, cooling rate, etc. Various polymorphs of clopidogrel hydrogen sulfate have been disclosed in different patents that are referred herein.

U.S. Pat. No. 6,429,210, (referred herein as '210), discloses two polymorphs of clopidogrel hydrogen sulfate referred to as form I and form II. The U.S. Pat. No. '210 identified that the precipitation method described in U.S. Pat. No. 4,847,265 has lead to crystalline form I. The '210 also deals with a new crystalline form IT of clopidogrel hydrogen sulfate. According to '210 patent both polymorphs were prepared from same solvent viz; acetone.

U.S. Pat. No. 6,767,913 discloses crystalline forms III, IV and V and amorphous form of clopidogrel hydrogen sulfate and processes for preparing the same. Amorphous form of clopidogrel hydrogen sulfate is prepared by dissolving clopidogrel hydrogen sulfate in an alcohol selected from the group consisting of methanol and ethanol followed by addition of ether as an anti solvent. Alternatively amorphous clopidogrel hydrogen sulfate is prepared by dissolving clopidogrel base in acetone, treated with sulfuric acid and refluxed, thereafter solvent is distilled out completely to obtain amorphous product.

PCT application WO 2004/020443 discloses process for the preparation of crystalline forms I of clopidogrel hydrogen sulfate. According to this patent application, a process is claimed to produce form I consistently by forming hydrogen sulfate salt of clopidogrel from a solvent selected from the series of C₁-C₅ alcohols or their esters with C₁-C₄ acids, optionally of mixture of alcohols and esters. The process involves dissolving clopidogrel base in solvents like isopropyl alcohol and/or butyl acetate, cooling the mixture, adding sulfuric acid and inoculating the mixture with form I of clopidogrel hydrogen sulfate and stirring the crystallized mixture precisely at a temperature between −5 and +15° C. to get crystals of clopidogrel in form I. In another process variant, the clopidogrel hydrogen sulfate was directly dissolved at reflux in the above mentioned solvents and crystallized under cooling.

Although the process mentioned in the above patent application worlds in butyl acetate, which is known to have hazardous properties (affects central nervous system and exposure limit is 150 ppm), but fails to give pure form I in other industrially friendly solvents like ethyl acetate under the specified conditions. As the form T is thermodynamically unstable, the process variant of dissolving clopidogrel hydrogen sulfate salt in solvents at higher temperature and cooling to precipitate form I resulted in form II or form IV or their mixture with form I.

PCT publication WO 2004/048385 discloses process for preparation of crystalline form I of clopidogrel hydrogen sulfate by reacting (S)-(+)clopidogrel base with concentrated sulfuric acid and the product is precipitated using solvent such as aliphatic or cyclic ethers or isobutyl methyl ketone.

We have found that the above process does not give pure form I in isobutyl methyl ketone under specified reaction conditions. There is formation of lumps and it is very difficult to isolate pure form I of clopidogrel hydrogen sulfate.

PCT application WO 2005/063708 A2 discloses processes for the preparation of hydrated form of amorphous clopidogrel hydrogen sulfate by treating clopidogrel base with sulfuric acid in suitable solvent like alcohols, tetrahydrofuran, dimethylformamide etc. and water, removing the solvent and isolating the amorphous form by adding antisolvent like pentane, hexane, heptane, cyclohexane, pet ether or mixture thereof.

PCT application WO 2005/003139 discloses process for preparation of form I of clopidogrel hydrogen sulfate wherein clopidogrel base is dissolved in solvent such as acetone, methylene chloride or isopropanol and treated with sulfuric acid, thereafter the reaction mixture is added to the suspension of clopidogrel hydrogen sulfate form I in di-isopropyl ether.

PCT application WO 2005/012300 discloses a process for the preparation of form I of clopidogrel hydrogen sulfate, by contacting clopidogrel bisulfate with sulfuric acid solution in acetate solvent followed by its isolation.

PCT application WO 2005/100364 discloses process for the preparation of Form I of Clopidogrel hydrogen sulfate, comprising converting clopidogrel salt into its base, said base is brought into dry solution with an organic solvent selected from methyl acetate, ethyl acetate, dichloromethane, or t-butyl methyl ether, followed by reaction with sulfuric acid.

PCT application WO 2005/104663 A2 discloses processes for the preparation of form I and form II of clopidogrel hydrogen sulfate by using ethyl acetate under different crystallization temperature. Form I is prepared by dissolving clopidogrel base in ethyl acetate and treated with sulfuric acid at 18-20° C. and maintaining the reaction temperature at 28-30° C. for 7-8 hours. Form II is prepared by dissolving clopidogrel base in ethyl acetate and cooling the reaction mass to 5-10° C., treated with sulfuric acid at same temperature and maintaining the reaction temperature at 10-15° C. for 8-12 hours.

US patent Application 2006/0100231 A1 disclose processes for the preparation of the amorphous clopidogrel hydrogen sulfate by dissolving clopidogrel base in methanol or ethanol or mixture thereof, acidifying it with sulfuric acid and subsequently slowly removing the solvent.

PCT Application WO 2005/003138 A2 discloses a process for the preparation of amorphous clopidogrel hydrogen sulfate which comprises dissolving clopidogrel base in acetone or dichloromethane, adding sulfuric acid or a mixture of sulfuric acid and diisopropyl ether, cyclohexane or ethyl acetate to the mixture, adding the obtained mixture containing clopidogrel hydrogen sulfate to the above solvent, and filtering optionally washing and drying the obtained precipitate.

So it is evident from the prior art that same solvent can give different polymorphs under different experimental conditions. A large number of factors can influence crystal nucleation and growth during this process, including the composition, the crystallization medium and the processes used to generate supersaturation and promote crystallization. The most notable variables of composition and processing are solvent/solvent combinations, degree of supersaturation, pH value, heating rate, cooling rate, etc. Various polymorphs of clopidogrel hydrogen sulfate have been disclosed in different patents/applications that are discussed above.

Most of the prior art methods for the preparation of form I of clopidogrel hydrogen sulfate from different solvents require very specific temperature range and specific conditions for getting reproducible results. Most of the methods are poorly reproducible, necessitating the optimization of experimental conditions along with that of the selection of solvents. Since form I is kinetically controlled and form II is thermodynamically controlled form, minor variation(s) in condition(s) appears to give form II instead of expected form I or a mixture of form I & form II. Hence, there is an urgent need to develop an efficient process which can give highly pure form I consistently without contamination of other forms.

Similarly most of the prior art methods for the preparation of amorphous clopidogrel hydrogen sulfate involve the use of ether solvents which are usually not recommended at industrial scale. However, there still remains a need for novel and modified process of preparing pure amorphous clopidogrel hydrogen sulfate which will be suitable for large-scale preparation, in terms of simplicity, chemical yield, consistency and purity of the product.

Thus the object of present invention is to provide a reliable method for obtaining pure polymorphs particularly form I and amorphous of clopidogrel hydrogen sulfate without detectable impurities of other forms.

Main object of present invention is to provide simple and efficient process for the preparation of highly pure form I and amorphous form of clopidogrel hydrogen sulfate.

### SUMMARY OF THE INVENTION

Accordingly, in one aspect, the present invention relates to an improved process for preparation of methyl(+)-(S)-α-(o-chlorophenyl)-6,7-dihydrothieno[3,2-c]pyridine-5(4H)-acetate hydrogen sulfate Form I of the formula I,

which comprises,
a) dissolving clopidogrel base in a suitable organic solvent,
b) adding halogenated solvent and seeding of form I of clopidogrel hydrogen sulfate,
c) cooling the reaction mixture to −10 to 0° C.;
d) adding solution of sulfuric acid in a suitable organic solvent at −10 to 0° C.;
e) stirring the reaction mixture for sufficient time to convert to form I of clopidogrel hydrogen sulfate;
f) isolating clopidogrel hydrogen sulfate form I.

In another aspect, the present invention relates to a simple and efficient process for the preparation of the amorphous form of clopidogrel hydrogen sulfate of formula I, which comprises dissolving clopidogrel base in a mixture of ketonic solvent and halogenated solvent, adding a mixture of sulfuric acid with ketonic solvent at low temperature, maintaining the reaction solution below ambient temperature for sufficient time to complete precipitation and isolating the pure amorphous clopidogrel hydrogen sulfate.

In further aspect, the present invention relates to one step process for the preparation of the amorphous form of clopidogrel hydrogen sulfate which comprises dissolving clopidogrel camphor sulfonic acid salt in halogenated solvent basifying it with a suitable base preferably sodium bicarbonate to yield clopidogrel base, dissolving the clopidogrel base in a mixture of ketonic solvent and halogenated solvent, adding a mixture of sulfuric acid with ketonic solvent at low temperature, maintaining the reaction solution below ambient temperature for sufficient time to complete precipitation and isolating the pure amorphous clopidogrel hydrogen sulfate.

### BRIEF DESCRIPTION OF THE DRAWINGS

FIG. 1 is a PXRD pattern for clopidogrel hydrogen sulfate form I

FIG. 2 is a DSC thermo gram of clopidogrel hydrogen sulfate I

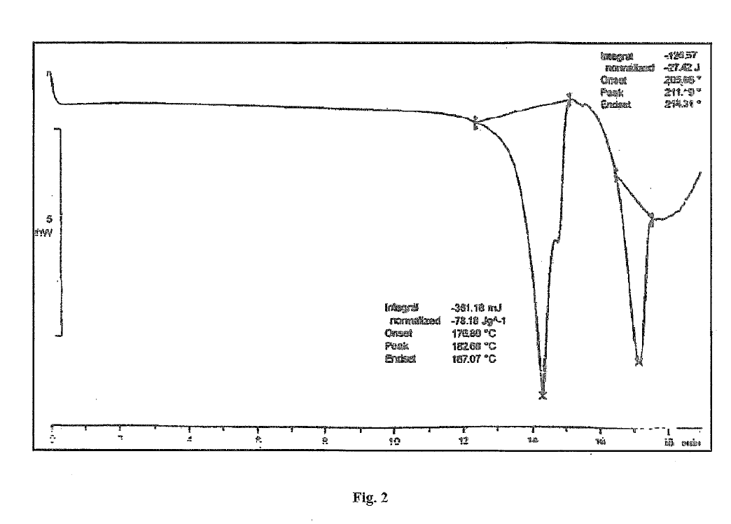

FIG. 3 is a PXRD pattern for amorphous clopidogrel hydrogen sulfate.

### DETAILED DESCRIPTION OF THE INVENTION

More particularly, the present invention describes improved process for the preparation of highly pure crystalline form I and amorphous forms of clopidogrel hydrogen sulfate. The process eliminate the problems of prior art and is convenient to operate for regular commercial production.

In one embodiment of the present invention, the clopidogrel base or clopidogrel camphor sulphonic acid salt used as the starting materials in the present invention, can be prepared by the methods known in prior art and described herein for reference. Generally, thieno[3,2-c]pyridine hydrochloride is reacted with α-bromo-(2-chlorophenyl)acetic acid methyl ester in N,N-dimethylformamide at 15-20° C. After completion of the reaction, water is added into the reaction mass and the product is extracted in methylene chloride which is distilled off to give (±) clopidogrel base as residue. The racemic mixture of clopidogrel so obtained is taken in acetone and 1 (−) camphor sulfonic acid is added and the reaction mass is refluxed for 4 hours. After that the reaction mass is maintained at 40-45° C. for 16 hours. Clopidogrel camphor sulfonic acid salt which separates out, is filtered, washed with acetone and dried.

Clopidogrel camphor sulfonic acid salt obtained is purified in acetone at reflux temperature followed by cooling to 20-25° C. The crystallized product is filtered, washed with acetone and dried. Quantity of N,N-dimethylformamide was 4.8 times that of thieno[3,2-c]pyridine hydrochloride in the above said reaction of thieno[3,2-c]pyridine hydrochloride with α-bromo-(2-chlorophenyl)acetic acid methyl ester. (+) Clopidogrel camphor sulfonic acid salt is then basified with aqueous sodium bicarbonate and extracted in methylene chloride which is distilled off to get (+) clopidogrel base as residue.

In another embodiment of the present invention, clopidogrel base formed by above process is converted to crystalline form I of clopidogrel hydrogen sulfate by taking clopidogrel base in a suitable organic solvent followed by addition of halogenated solvent at −10 to 0° C. This is followed by addition of sulfuric acid in a suitable solvent and isolation of the required product in high yield.

Preferably, clopidogrel base is taken in suitable organic solvent and halogenated solvent is added to it at ambient temperature. The solution is stirred to get a clear solution which is cooled to −10 to 0° C.

In a preferred embodiment, clopidogrel base is taken in suitable organic solvent at ambient temperature followed by cooling to −10 to 0° C. To the cooled solution, halogenated hydrocarbon is added. Thus, cooling the reaction mixture before or after the addition of the halogenated solvent does not affect the course of reaction and in any way does not limit the scope of invention.

In a preferred embodiment, suitable organic solvent is selected from ketonic and aliphatic hydrocarbon solvent. Preferably, the ketonic solvent is selected of a group of solvents which have the ability to dissolve clopidogrel base completely thus includes methyl isobutyl ketone. The aliphatic hydrocarbon solvent can be selected from amongst n-hexane and n-heptane. Halogenated solvent referred herein is selected from methylene dichloride, chloroform, carbontetrachloride, ethylene dichloride. Preferably, methylene chloride is used.

The reaction mixture is seeded with crystalline form I of clopidogrel hydrogen sulfate while maintaining the temperature at −10 to 0° C. Seeding of polymorph form I is essential for the preparation of pure form I of clopidogrel hydrogen sulfate.

Thereafter reaction mixture is treated with a solution of sulfuric acid in a suitable organic solvent preferably ketonic solvent or aliphatic hydrocarbon at a temperature of −10 to 0° C. The solution of sulfuric acid in organic solvent is prepared by dropwise addition of sulfuric acid in organic solvent at −10 to 0° C.

According to the present invention, it is advantageous to add halogenated solvent to the reaction mixture before adding sulfuric acid because addition of halogenated solvent makes the reaction mixture free flowing without any lumps.

The resulting reaction mixture is stirred further at temperature below 0° C. for about two to six hours followed by increase in temperature slowly in about more than six hours to 15-20° C. The reaction mixture is again stirred for additional eight to twelve hours at 15-20° C. to prepare pure form I. Thereafter pure product is recovered by filtration at 15-20° C. and further dried to remove solvents in a vacuum tray drier. The clopidogrel hydrogen sulfate form I so obtained, is highly pure without detectable impurities of form II of clopidogrel hydrogen sulfate or any other form in X-ray diffraction pattern.

In yet another embodiment, the present invention describes modified and novel process for the preparation of pure amorphous clopidogrel hydrogen sulfate. The present invention relates to the synthesis of clopidogrel hydrogen sulfate starting from clopidogrel base. Generally, clopidogrel base is taken in a mixture of ketonic solvent and halogenated solvent at ambient temperature. The reaction mixture is cooled and treated with a solution of sulfuric acid in ketonic solvent and stirred for sufficient time to prepare amorphous clopidogrel hydrogen sulfate which is isolated by filtration.

In a preferred embodiment, ketonic solvent is preferably selected from a group of ketonic solvents which have the ability to dissolve clopidogrel base completely thus includes methyl isobutyl ketone. Halogenated solvent referred herein is selected from methylene dichloride, chloroform, carbon tetrachloride, ethylene dichloride. It is advantageous to add halogenated solvent in ketonic solvent to avoid lump formation. Addition of halogenated solvent thus makes the solution free flowing without any lumps. The solution of sulfuric acid in ketonic solvent is prepared by drop wise addition of sulfuric acid in ketonic solvent at low temperature.

Preferably clopidogrel base is taken in a mixture of methyl isobutyl ketone and methylene chloride at ambient temperature. The resulting solution is cooled to a temperature of −7 to −2° C. To the chilled reaction mixture, a solution of sulfuric acid in methyl isobutyl ketone is added at a temperature of −7 to −2° C. The resulting reaction mixture is stirred at −7 to −2° C. for about two to four hours followed by raising the temperature slowly at 15-20° C. The reaction mixture is stirred for sufficient time to precipitate completely pure amorphous clopidogrel hydrogen sulfate. It usually takes eight to twelve hours at 15-20° C. to precipitate amorphous product completely. The product is isolated by well known methods in prior art i.e. by filtration. The filtered product is dried under vacuum to remove solvent and isolate pure amorphous clopidogrel hydrogen sulfate in high yield and purity.

In another embodiment of the present invention discloses a one step process for the preparation of pure amorphous clopidogrel hydrogen sulfate from clopidogrel camphor sulfonic acid salt of formula II:

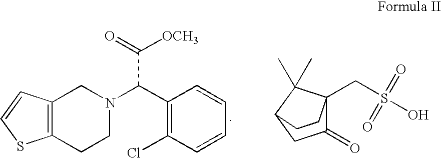

The clopidogrel camphor sulfonic acid salt, used as the starting material of the present invention, can be prepared by the methods well known in prior art.

Generally, clopidogrel camphor sulfonic acid salt is dissolved in halogenated solvent and treated with a solution of suitable base to hydrolyze the salt. After layer separation, organic solvent is distilled out to obtain clopidogrel base as residue. The residue is dissolved in a mixture of suitable ketonic and halogenated solvent and cooled to temperature below 0° C. This is followed by addition of solution of sulfuric acid in ketonic solvent and stirred for sufficient time to complete precipitation of amorphous clopidogrel hydrogen sulfate which is isolated by filtration. The ketonic solvent is preferably selected from a group of ketonic solvents which have the ability to dissolve clopidogrel base completely thus includes methyl isobutyl ketone. Halogenated solvent referred herein is selected from methylene dichloride, chloroform, carbon tetrachloride, ethylene dichloride.

Specifically clopidogrel camphor sulfonic acid salt is prepared by reacting 4,5,6,7-tetrahydrothieno[3,2-c]pyridine hydrochloride with α-bromo-(2-chlorophenyl)acetic acid methyl ester in N,N-dimethylformamide at 15-20° C. After reaction completion, water is added into the reaction mass and the product is extracted in methylene chloride which is distilled off to give (±) clopidogrel base as residue. The racemic mixture of clopidogrel so obtained is taken in acetone and 1 (−) camphor sulfonic acid is added and the reaction mass is refluxed for 4 hours. After that the reaction mass is maintained at 40-45° C. for 16 hours. Clopidogrel camphor sulfonic acid salt separated is filtered, washed with acetone, and dried. Optionally clopidogrel camphor sulfonic acid salt obtained is purified in acetone at reflux followed by cooling to 20-25° C. (+) Clopidogrel camphor sulfonic acid salt is then taken in methylene chloride and basified with aqueous sodium bicarbonate and extracted in methylene chloride which is distilled off to get (+) clopidogrel base as residue. Preferably clopidogrel base is taken in a mixture of methyl isobutyl ketone and methylene chloride at ambient temperature. The resulting solution is cooled to temperature of −7 to −2° C. To the chilled reaction mixture, a solution of sulfuric acid in methyl isobutyl ketone is added at a temperature of −7 to −2° C. The resulting reaction mixture is stirred at −7 to −2° C. for about two to four hours followed by raising the temperature slowly at 15-20° C. The reaction mixture is stirred for sufficient time to precipitate completely pure amorphous clopidogrel hydrogen sulfate. It usually takes eight to twelve hours at 15-20° C. to precipitate amorphous product completely. The product is isolated by well known methods in prior art i.e. by filtration. The filtered product is dried under vacuum to remove solvent and isolate pure amorphous clopidogrel hydrogen sulfate in high yield and purity.

The present invention meets the need in the art for improved, low cost and environment friendly process for preparing form I and amorphous clopidogrel hydrogen sulfate. The present invention is advantageous over the prior art processes being robust resulting consistently in single form and free from any other polymorphs of clopidogrel hydrogen sulfate. The process can be scaled-up easily conveniently and inexpensively for industrial large-scale production.

The present invention is further illustrated by the following examples which are provided merely to be exemplary of the inventions and is not intended to limit the scope of the invention. Certain modifications and equivalents will be apparent to those skilled in the art and are intended to be included within the scope of the present invention.

### EXAMPLES

### Example 1

### Step-I: Preparation of (+) Clopidogrel Camphor Sulfonic Acid Salt

N,N-Dimethylformamide (4.80 L) was cooled to 15-20° C. and added to triethylamine (1.261) and thieno[3,2-c]pyridine hydrochloride (1.0 kg) under stirring. To the reaction mass α-bromo-2-chlorophenyl acetic acid methyl ester (1.65 kg) was added slowly followed by stirring for 30 minutes at 15-20° C. It was followed by addition of water (4.0 l) and methylene chloride (4.0 l). The layers were separated and methylene chloride was distilled out under reduced pressure to afford (±) clopidogrel free base as residue (1.83 kg). The racemic mixture of clopidogrel so obtained was taken in acetone and 1 (−) camphor sulfonic acid (1.21 kg) was added. The reaction mass was refluxed for 4 hours and maintained at 40-45° C. for 16 hours. Clopidogrel camphor sulfonic acid salt obtained was purified in 21 volumes of acetone at reflux temperature followed by cooling to 20-25° C. The crystallized product was filtered, washed with acetone and dried to afford the title compound.

### Step-II: Preparation of Clopidogrel Base

The (+) clopidogrel camphor sulfonic acid salt obtained in above step (0.84 kg), was basified with aqueous sodium bicarbonate (0.42 kg) using methylene chloride (5.04 L) as reaction medium. The product was extracted with methylene chloride. Methylene chloride was distilled off to afford 0.5 kg of the title compound.

### Example 2

### Preparation of Clopidogrel Hydrogen Sulfate Form I

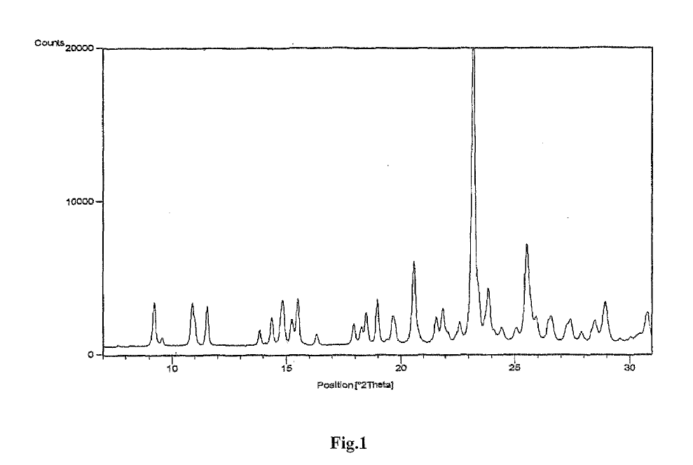

Clopidogrel base (28 g) was dissolved in methyl isobutyl ketone (345 ml) at room temperature. To this solution, chloroform (4.5 ml) and seed of Clopidogrel Form I (5.6 g) were added and the reaction mixture was cooled to −10 to −5° C. To the reaction mass, a cooled solution of sulfuric acid (7.2 g) in methyl isobutyl ketone (173 ml) was added drop wise maintaining temperature below 0° C. under inert atmosphere. The reaction mixture was stirred at same temperature for 3 hours. Thereafter the temperature of reaction mixture was raised to 15-17° C. slowly and stinting was continued for further 10 hours. The product was filtered, washed with methyl isobutyl ketone and dried to obtain the title compound. The powdered X-ray diffraction pattern shown in FIG. 1 indicated that the material obtained is form I of clopidogrel hydrogen sulfate.

### Example 3

### Preparation of Clopidogrel Hydrogen Sulfate Form I

Clopidogrel base (28 g) was dissolved in methyl isobutyl ketone (345 ml) at room temperature. To this solution, carbon tetrachloride (4.5 ml) was added and the reaction mixture was seeded with clopidogrel form T (5.6 g). The solution was cooled to −10 to −5° C. To the reaction mass, a cooled solution of sulfuric acid (7.2 g) in, methyl isobutyl ketone (173 ml) was added drop wise under inert atmosphere at −10 to −5° C. The reaction mixture was stirred at same temperature for 3 hours. Thereafter the temperature of reaction mixture was raised to 16-18° C. slowly and stirring was continued for further 10 hours. The product was filtered, washed with methyl isobutyl ketone and dried to obtain the title compound.

### Example 4

### Preparation of Clopidogrel Hydrogen Sulfate Form I

Clopidogrel base (28 g) was dissolved in methyl isobutyl ketone (345 ml) at room temperature and the solution was cooled to −10 to 0° C. To this solution, ethylene dichloride (4.5 ml) was added and the reaction mixture was seeded with clopidogrel form I (5.6 g). To the reaction mass, a cooled solution of sulfuric acid (7.2 g) in methyl isobutyl ketone (173 ml) was added drop wise under inert atmosphere at −10 to −5° C. The reaction mixture was stirred at same temperature for 3 hours. Thereafter the temperature of reaction mixture was raised to 16-18° C. slowly and stirring was continued for further 10 hours. The product was filtered, washed with methyl isobutyl ketone and dried to obtain the title compound.

### Example 5

### Preparation of Clopidogrel Hydrogen Sulfate Form I

Clopidogrel base (15 g) was dissolved in n-hexane (185 ml) at room temperature and the solution was cooled to −10 to −5° C. To this solution, methylene chloride (2.5 ml) was added and the reaction mixture was seeded with clopidogrel form I (3.0 g). To the reaction mass, a cooled solution of sulfuric acid (7.2 g) in n-hexane (93 ml) was added drop wise under inert atmosphere at −10 to −5° C. The reaction mixture was stirred at same temperature for 3 hours. Thereafter the temperature of reaction mixture was raised to 16-18° C. slowly and stirring was continued for further 10 hours. The product was filtered, washed with n-hexane and dried to obtain pure clopidogrel hydrogen sulfate form I.

### Example 6

### Preparation of Clopidogrel Hydrogen Sulfate Form I

Clopidogrel base (20 g) was dissolved in n-heptane (246 ml) at room temperature and the solution was cooled to −10 to −5° C. To this solution, methylene chloride (3.2 ml) was added and the reaction mixture was seeded with clopidogrel form I (4.0 g). To the reaction mass, a cooled solution of sulfuric acid (7.2 g) in n-hexane (93 ml) was added drop wise under inert atmosphere at −10 to −5° C. The reaction mixture was stirred at same temperature for 3 hours. Thereafter the temperature of reaction mixture was raised to 17° C. slowly and stirring was continued for further 10 hours. The product was filtered, washed with n-heptane and dried to obtain pure clopidogrel hydrogen sulfate form I.

### Example 7

### Preparation of Clopidogrel Hydrogen Sulfate Form I

Clopidogrel camphor sulphonic acid salt (4.0 kg) was taken in methylene chloride (24.0 l) and the aqueous solution of sodium bicarbonate (2.0 kg) was added. After complete addition the layers were separated and methylene chloride was distilled off to obtain clopidogrel base (2.4 kg) as residue. The residue was dissolved in methyl isobutyl ketone (29.88 l) at room temperature and the solution was cooled to −10 to −5° C. To this solution, methylene chloride (0.4 l) was added and the reaction mixture was seeded with clopidogrel form I (0.48 kg). To the reaction mass, a cooled solution of sulfuric acid (0.64 kg) in methyl isobutyl ketone (15.0 l) was added drop wise under inert atmosphere at −10 to −5° C. The reaction mixture was stirred at same temperature for 3 hours. Thereafter the temperature of reaction mixture was raised to 17° C. slowly and stirring was continued for further 10 hours. The product was filtered, washed with methyl isobutyl ketone and dried under vacuum to obtain pure clopidogrel hydrogen sulfate form I.

### Example 8

### Preparation of Amorphous Clopidogrel Hydrogen Sulphate

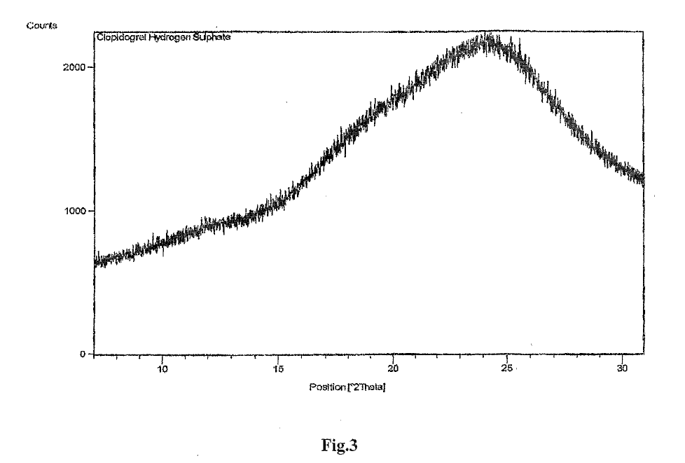

Clopidogrel free base (53.2 g) was dissolved in methyl isobutyl ketone (709.6 ml) and methylene chloride (9.5 ml). The reaction mixture was cooled to −7 to −2° C. and a solution of sulfuric acid (15.2 g) in methyl isobutyl ketone (357.2 ml) was added at −7 to −2° C. The reaction mass was stirred for 3 hours at same temperature. Thereafter, the temperature was raised to 15-20° C. and the reaction mass was further stirred for 10 hours. The product precipitated out was filtered under inert atmosphere, washed with methyl isobutyl ketone (3×95 ml) and dried under vacuum at 50-55° C. to yield 66.5 g of title compound. Powdered X-ray diffraction pattern shows no peaks as shown in FIG. 3, thus demonstrating that material is amorphous.

### Example 9

### Preparation of Amorphous Clopidogrel Hydrogen Sulphate

Clopidogrel camphor sulfonic acid salt (95 g) was dissolved in methylene dichloride (570 ml) and to this solution; an aqueous solution of sodium bicarbonate was added at 15-20° C. till pH 7-8. The organic layer was separated and methylene dichloride was distilled out to yield clopidogrel free base as residue. Clopidogrel free base obtained (53.2 g) was dissolved in methyl isobutyl ketone (709.6 ml) and methylene dichloride (9.5 ml). The reaction mass was cooled to −7 to −2° C. and a solution of sulfuric acid (15.5 g) in methyl isobutyl ketone (357.2 ml) was added at −7 to −2° C. The reaction mass was further stirred for 3 hours at same temperature. Thereafter, the temperature was raised to 17±2° C. and the reaction mass was further stirred for 10 hours. The product precipitated out was filtered under inert atmosphere, washed with methyl isobutyl ketone (3×95 ml) and dried to yield 66.5 g of title compound.

We claim:

1. A process for the preparation of methyl(+)-(S)-α-(o-chlorophenyl)6,6-dihydrothieno[3,2c]pyridine-5(4H)-acetate hydrogen sulfate (clopidogrel hydrogen sulfate) form I, which comprises,
dissolving clopidogrel base in suitable organic solvent selected from ketones and aliphatic hydrocarbons,
adding halogenated solvent and seeding of form I of clopidogrel hydrogen sulfate,
cooling the reaction mixture to −10 to 0° C.,
adding solution of sulfuric acid in suitable organic solvent maintaining the temperature below 0° C.,
stirring the reaction mixture for sufficient time to convert to form I of clopidogrel hydrogen sulfate,
isolating clopidogrel hydrogen sulfate form I.

2. A process according to claim 1 wherein ketones and aliphatic hydrocarbons are methyl isobutyl ketone, n-hexane and n-heptane.

3. A process according to claim 1 wherein halogenated solvent is selected from methylene chloride, ethylene dichloride, chloroform and carbon tetrachloride.

4. A process according to claim 1 wherein halogenated solvent is preferably methylene chloride.

5. A process according to claim 1 wherein clopidogrel hydrogen sulfate form I having a powder X-ray diffraction pattern as shown in FIG. 1.

6. A process for the preparation of highly pure clopidogrel hydrogen sulfate form I substantially the same as described herein.

7. A process for the preparation of clopidogrel hydrogen sulfate form I, which comprises, suspending clopidogrel camphor sulphonic acid salt in organic solvent, treating the reaction mass with aqueous solution of sodium bicarbonate, distilling the organic layer to obtain clopidogrel base as residue, dissolving clopidogrel base in suitable organic solvent, adding halogenated solvent and seeding of form I of Clopidogrel hydrogen sulfate, cooling the reaction mixture to −10 to 0° C., adding solution of sulfuric acid in suitable organic solvent maintaining the temperature below 0° C., stirring the reaction mixture for sufficient time to convert to form I of clopidogrel hydrogen sulfate, isolating clopidogrel hydrogen sulfate form I.

8. A process for the preparation of amorphous clopidogrel hydrogen sulfate of formula I, which comprises:
dissolving clopidogrel base in a mixture of ketonic solvent and halogenated solvent;
cooling the reaction mass to below 0° C.,
adding solution of sulfuric acid in ketonic solvent,
raising the reaction temperature to 15-20° C.,
stirring the reaction mass for sufficient time to precipitate amorphous product and isolating amorphous clopidogrel hydrogen sulfate.

9. A process according to claim 8, wherein ketonic solvent is preferably selected of a group of ketonic solvents which have the ability to dissolve clopidogrel base completely.

10. A process according to claim 8, wherein ketonic solvent is methyl isobutyl ketone.

11. A process according to claim 8, wherein halogenated solvent is selected from methylene dichloride, chloroform, carbon tetrachloride, ethylene dichloride.

12. A process according to claim 8, wherein sulfuric acid is added at −7 to −2° C.

13. A process for the preparation of amorphous clopidogrel hydrogen sulfate of formula I, which comprises:
dissolving clopidogrel camphor sulfonic acid salt in halogenated solvent;
adding a suitable base to the reaction mixture;
distilling off solvent to yield clopidogrel base as residue;
dissolving clopidogrel base in a mixture of ketonic solvent and halogenated solvent;
cooling the reaction mass to below 0° C.;
adding solution of sulfuric acid in ketonic solvent,
raising the reaction temperature to 15-20° C.;
isolating amorphous clopidogrel hydrogen sulfate.

14. A process according to claim 13, wherein suitable base used is preferably sodium bicarbonate.

15. A process according to claim 13, wherein ketonic solvent is methyl isobutyl ketone.

16. A process according to claim 13, wherein halogenated solvent is methylene dichloride.
